(19) United States
(12) Patent Application Publication
(10) Pub. No.: US 2022/0387652 A1
(43) Pub. Date: Dec. 8, 2022

(54) TEMPORAL MOUNT DISINFECTION DEVICE

(71) Applicant: GHSP, Inc., Holland, MI (US)

(72) Inventors: Richard W. Harris, Grand Haven, MI (US); Ian P. Sage, Holland, MI (US); Aghuinyue Umenei, Grand Rapids, MI (US)

(21) Appl. No.: 17/834,473

(22) Filed: Jun. 7, 2022

Related U.S. Application Data
(60) Provisional application No. 63/197,950, filed on Jun. 7, 2021.

(51) Int. Cl. A61L 2/26; A61L 2/10

(52) U.S. Cl. CPC A61L 2/26; A61L 2/10

(57) ABSTRACT

A temporal mount disinfection device for providing portable UV-C disinfection includes an elongated housing having a plurality of apertures. A plurality of UV-C LEDs project light through each one of the plurality of apertures. A spot light emanates from the housing and is used for aiming the plurality of UV-C LEDs to a specific location within a vehicle. A mount is connected to the housing that is reconfigurable for allowing the disinfection device to be mounted in various locations throughout a vehicle.

### FIELD OF THE INVENTION

The present invention relates generally to air and surface disinfection and more particularly to a system for dual mode disinfection for vehicles.

### BACKGROUND

Applying a safe and useful dose of Ultra-Violet C (UV-C) light for disinfection within a vehicular environment presents several problems. These include the need to maintain the safety of an operator, or anyone in the vicinity of the UV-C. UV-C light in unsafe doses can present a hazard to the user and consequently excessive exposure must be avoided. Disinfection often can require providing an effective dose of UV-C radiation to multiple surfaces within the vehicle for lengthy periods of time. Since UV-C disinfection is not portable, it can be difficult to use this technology to provide light based disinfection.

Thus, additional solutions are needed to safely and efficiently disinfect surfaces within vehicles that are in frequent contact with occupants of the vehicle.

### BRIEF DESCRIPTION OF THE FIGURES

The accompanying figures, where like reference numerals refer to identical or functionally similar elements throughout the separate views and which together with the detailed description below are incorporated in and form part of the specification, serve to further illustrate various embodiments and to explain various principles and advantages all in accordance with the present invention.

FIG. 1 is perspective view of a temporal mount disinfection device according to an embodiment of the invention.

FIG. 2 is a perspective view of a plurality of temporal mount disinfection devices configured within at different locations within a vehicle.

FIG. 3A and FIG. 3B are perspective views showing the disinfection device configured with key chain and extension pole according to an embodiment of the invention.

FIG. 4 is a perspective view a top half of the housing of the disinfection device where the top half of the housing is pivotable.

FIG. 5A, FIG. 5B and FIG. 5C are perspective view illustrating a mount used with the disinfection device that is adjustable into various configurations to accommodate its mounting position.

FIG. 6A, FIG. 6B, FIG. 6C and FIG. 6D illustrate various mounting locations within a vehicle for the disinfection device.

FIG. 7A and FIG. 7B illustrate the disinfection device housed in various sizes with differing number of disinfection LEDs.

FIG. 8A and FIG. 8B are magnified views illustrating a disinfection device arranged using various sun visor configurations.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

### DETAILED DESCRIPTION

Before describing in detail embodiments that are in accordance with the present invention, it should be observed that the embodiments reside primarily in an apparatus providing a temporal mount disinfection device. Accordingly, the apparatus components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

In this document, relational terms such as first and second, top and bottom, and the like may be used solely to distinguish one entity or action from another entity or action without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “comprises . . . a” does not, without more constraints, preclude the existence of additional identical elements in the process, method, article, or apparatus that comprises the element.

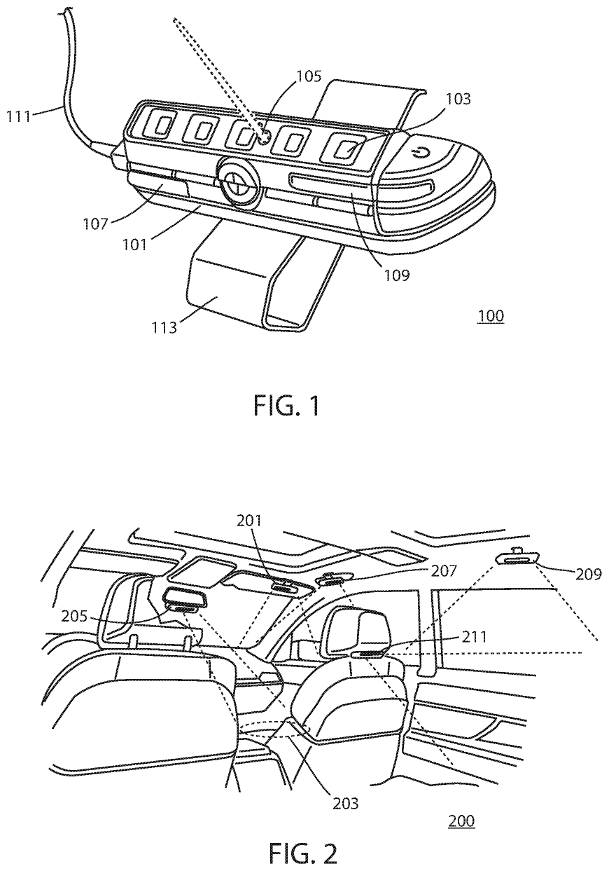

FIG. 1 is perspective view of a temporal mount disinfection device 100 according to an embodiment of the invention. The disinfection device 100 is portable and satisfies the need for flexibility in mounting the device in multiple locations within a vehicle for efficient disinfection. The embodiment as described herein works to eliminate line of sight issues for the UV light as the device can be placed at practically at any desired location without light obstruction.

The temporal mount disinfection device 100 includes a housing 101 that is configured to hold a plurality of UV-C LEDs 103. In this embodiment, the UV-C LEDs are linearly aligned across the face of the housing 101 and use a red LED aiming light 105 to more easily direct the UV-C disinfection light to a desired location. The aiming light or “spotlight feature” utilizes an indicator LED for properly mounting of a globe or “G-type” LED module by the consumer as an auto accessory. Depending on the mounting location, the spotlight can be used at start of operation, to help users adjust the disinfection device to a specific and/or proper location for disinfection.

In use, a procedure to spotlight the disinfection device becomes a security feature for the system, especially with children, to prevent inadvertent turn ON of device. Moreover, an indicator light 107 is used to provide indicia that the UV-C light is in an “on” state. The bezel 109 further aids in indicating to the user when the UV-C LEDs are activated. A charging cable 111 can be used to power and/or charge internal batteries within the housing and to power the device when 12-vdc power is continuously available. As seen in the figures hereinafter, a mount or clip 113 is used to mount the portable device at desired locations thought-out the vehicle.

FIG. 2 is a perspective view illustrating a plurality of temporal mount disinfection devices configured at different locations within a vehicle interior 200. The illustration shows the utility of the mounting bracket and how it can be used at multiple locations within the vehicle. For example, a disinfection device 201 can be mounted to a visor providing UV-C disinfection 203 at some area below its mounting location. Similarly, a disinfection device 205 can be mounted behind a seat headrest providing UV-C light to the floor and rear seat surfaces. A disinfection device 207 can be mounted to a front grab handle 207 and rear grab handle 209 for disinfect interior vehicle surfaces within its vicinity. Further, a disinfection device 211 can also be mounted under a head rest for disinfecting surfaces at the rear of the vehicle.

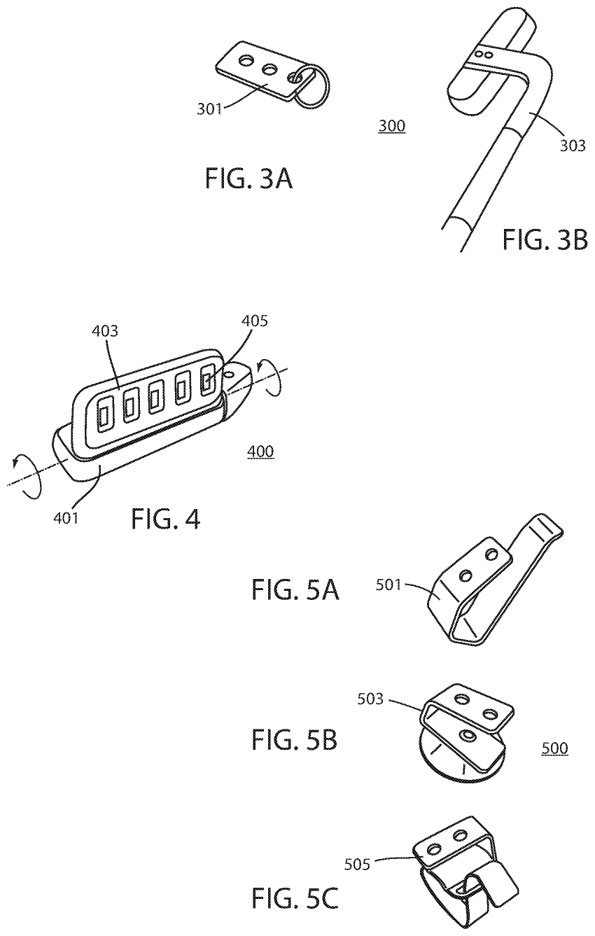

FIG. 3 is a perspective view showing the disinfection device configured as a key chain according to an embodiment of the invention. In one embodiment, the disinfection device 301 can be configured as a key chain so that it is typically present with the vehicle. In another embodiment, the disinfection device 303 can be configured with a wand or arm as a hand-held device. Thus, the use of the mounting clip can provide for a multi-use disinfection system outside of the vehicle with these types of transformable mount/holding options. This allows the disinfection device to operate as a multi-use disinfection system outside of vehicle with the transformable mount/holding option. Portability of the system implies it can be taken out of vehicle environment and used elsewhere for surface disinfection (camping, motorcycle, etc.)

FIG. 4 is a perspective view of an alternative embodiment of the disinfection device 400 where a top half of the housing, having UV-C LEDs used for disinfection, is pivotable. In this embodiment, the housing 401 has a top half 403 having a plurality of LED apertures 405. The top half 403 swings and/or pivots so to direct the UV-C light can be adjusted to specific disinfection locations. This hinged type of LED arrangement or “stack” can be used for adjustment of disinfection light direction.

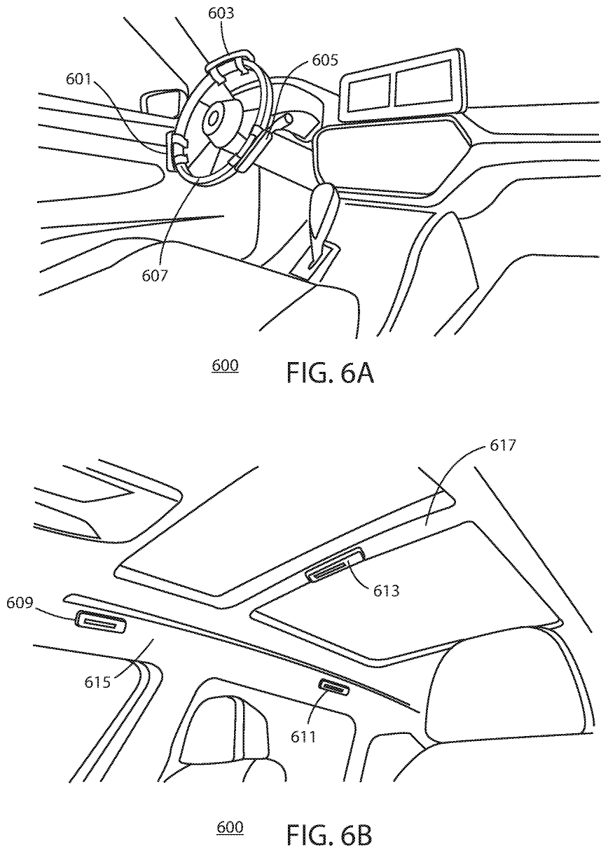

FIG. 5A, FIG. 5B and FIG. 5C illustrate various styles of clip mounts used with the disinfection device allowing the device to be easily adjustable into various configurations to accommodate its mounting position. FIG. 5A illustrates the mount can be configured for use with a strap 501. FIG. 5B illustrates the mount configured for use with a section cup 503. FIG. 5C illustrates the mount configured for used with a spring clip 505. Each of these embodiments allow the disinfection device to be mounted at different vehicle locations or within different types of vehicles.

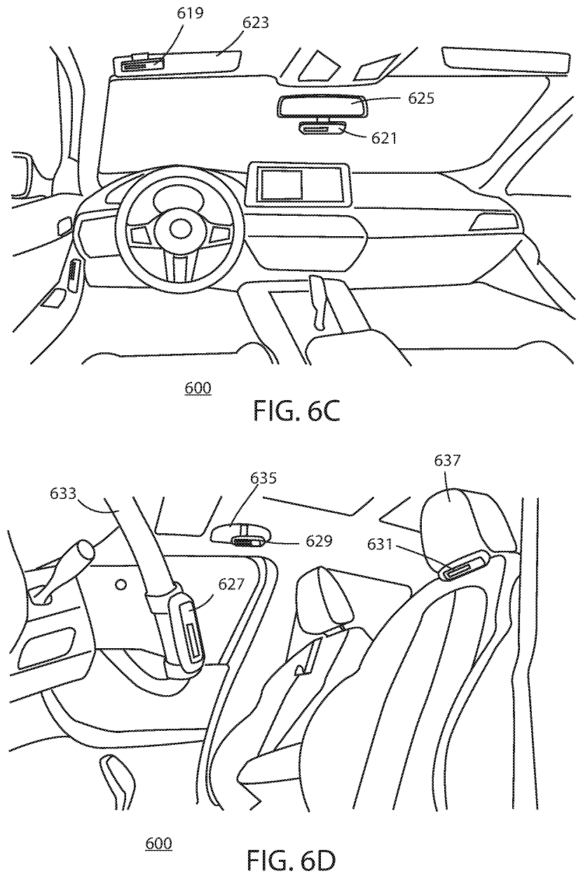

FIG. 6A, FIG. 6B, FIG. 6C and FIG. 6D illustrate additional mounting locations of the disinfection deice within a vehicle. More specifically, FIG. 6A is a perspective view illustrating a plurality of disinfection devices 601, 603, 605 mounted around a steering wheel 607. FIG. 6B illustrates a plurality of disinfection devices 609, 611, 613 mounted to a vehicle roof interior 615 and headliner 617. FIG. 6C illustrates a disinfection devices 619, 621 mounted to driver sun visor 623 and rear-view mirror 625. FIG. 6D illustrates various disinfection devices 627, 629, 631 mounted to the vehicle's steering wheel 633, rear view mirror 635 and under the driver's head rest 637.

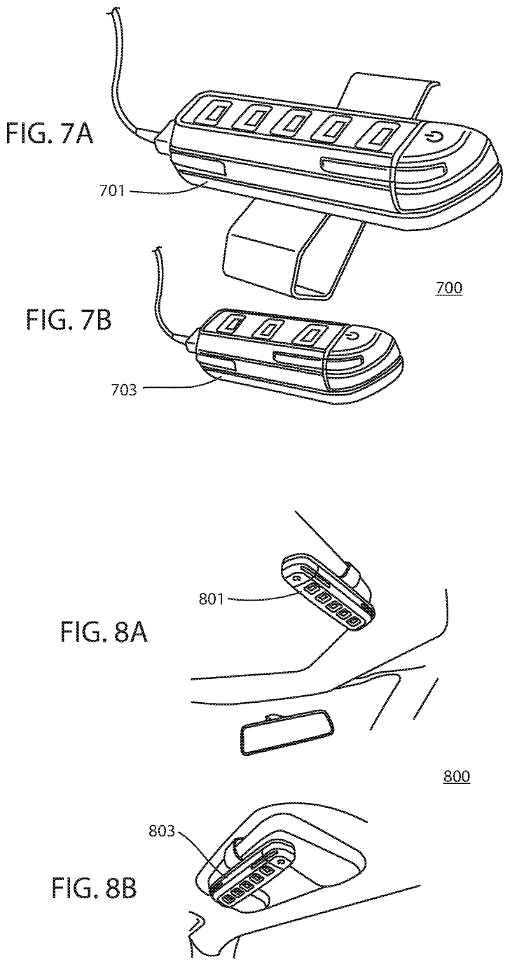

FIG. 7 illustrates an alternative embodiment where the disinfection device is configured in various sizes with differing number of disinfection LEDs. In this embodiment, the disinfection device 701 is a five (5) UV-C LED model while disinfection device 703 is a smaller three (3) UV-C LED model.

FIG. 8A and FIG. 8B are magnified views illustrating disinfection devices in various sun visor configurations. In FIG. 8A, the disinfection device 801 is attached to the front side of driver's side sun visor. In FIG. 8B, the disinfection device 803 is attached to a back side of the driver's side sun visor.

Thus, embodiments of the present invention are directed to a temporal mount disinfection device that includes an elongated housing having a plurality of apertures. A plurality of UV-C LEDs are used for projecting light through each one of the plurality of apertures. A spotlight is configured with in the housing for aiming the plurality of UV-C LEDs. The housing uses a reconfigurable mount for allowing the disinfection device to be mounted in various components throughout a vehicle.

In the foregoing specification, specific embodiments of the present invention have been described. However, one of ordinary skill in the art appreciates that various modifications and changes can be made without departing from the scope of the present invention as set forth in the claims below. Accordingly, the specification and figures are to be regarded in an illustrative rather than a restrictive sense, and all such modifications are intended to be included within the scope of present invention. The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

We claim:

1. A temporal mount disinfection device comprising:
an elongated housing having a plurality of apertures;
a plurality of UV-C LEDs for projecting light through each one of the plurality of apertures;
at least one spotlight emanating from the housing for aiming the plurality of UV-C LEDs; and
a reconfigurable mount connected to the housing for allowing the disinfection device to be mounted in various components throughout a vehicle.

2. A temporal mount disinfection device as in claim 1, further comprising:
at least one battery for powering the device.

3. A temporal mount disinfection device as in claim 1, wherein the battery is rechargeable.

4. A temporal mount disinfection device as in claim 1, further comprising:
at least one indicator light for annunciating an operating state of the device.

5. A temporal mount disinfection device as in claim 1, further comprising an opaque window for determining activation of the plurality of UV-C LEDs.

6. A temporal mount disinfection device as in claim 1, wherein the spotlight is configuration between the plurality of UV-C LEDs.

7. A temporal mount disinfection device as in claim 1, wherein the spotlight is a red LED.

8. A temporal mount disinfection device for use in a vehicle comprising:
an elongated housing having a plurality of apertures;
a plurality of UV-C LEDs, each one of the plurality of UV-C LEDs projecting disinfecting light through one of the plurality of apertures;
at least one LED spotlight emanating from the housing for aiming the plurality of UV-C LEDs to a specific vehicle interior location; and
a reconfigurable mount connected to the housing for allowing the disinfection device to be mounted to various vehicle components.

9. A temporal mount disinfection device as in claim 8, further comprising an indicator light for indicating the operational state of the plurality of UV-C LEDs.

10. A temporal mount disinfection device as in claim 9, where the indicator light is configured between the plurality of apertures.

11. A temporal mount disinfection device as in claim 8, where the plurality of UV-C LEDs are powered by a rechargeable battery within the housing.

12. A temporal mount disinfection device as in claim 8, wherein the spotlight provides red light.

13. A temporal mount disinfection device for use in disinfecting automotive interior surfaces comprising:
an elongated housing having a plurality of light apertures;
at least one UV-C LED configured to project light though one of the plurality of light apertures;
at least one spotlight emanating a light from the housing for aiming the plurality of UV-C LEDs to an interior surface location;
a reconfigurable mount connected to the housing for allowing the disinfection device to be mounted to various components throughout an interior of the vehicle;
an indicator light for indicating the operating status of the at least one UV-C LED; and
a rechargeable battery within the elongated housing for powering the at least one UV-C LED and at least one spotlight.

14. A temporal mount disinfection device as in claim 1, further comprising an opaque window for viewing activation of the plurality of UV-C LEDs.

15. A temporal mount disinfection device as in claim 1, wherein the spotlight is configured in-between the plurality of UV-C LEDs.

16. A temporal mount disinfection device as in claim 1, wherein the spotlight is a red LED.

17. A temporal mount disinfection device as in claim 1, wherein the at least one UV-C LED is five LEDs.
